(19) United States
(12) Patent Application Publication
(10) Pub. No.: US 2005/0208103 A1
(43) Pub. Date: Sep. 22, 2005

(54) Targeted transscleral controlled release drug delivery to the retina and choroid

(76) Inventors: Anthony P. Adamis, Jamaica Plain, MA (US); Evangelos S. Gragoudas, Lexington, MA (US); Joan W. Miller, Winchester, MA (US)

Correspondence Address: GOODWIN PROCTER LLP;PATENT ADMINISTRATOR, EXCHANGE PLACE, BOSTON, MA, 02109-2881, US

(21) Appl. No.: 11/124,949

(22) Filed: May 9, 2005

Related U.S. Application Data
(63) Continuation of application No. 09/478,099, filed on Jan. 5, 2000, now abandoned.
(60) Provisional application No. 60/114,905, filed on Jan. 5, 1999.

(51) Int. Cl. A61K 39/395; A61K 31/695

(52) U.S. Cl. 424/427; 424/143.1

(57) ABSTRACT

The invention provides methods for delivering a therapeutic or diagnostic agent to the eye of a mammal. The method involves contacting sclera with a therapeutic or diagnostic agent so as to permit its passage through the sclera into the choroidal and retinal tissues. The sclera may be contacted with a therapeutic or diagnostic agent together with a device for enhancing transport of the agent through the sclera.

### CROSS REFERENCE TO RELATED APPLICATIONS

This application is a continuation-in-part of U.S. Ser. No. 60/114,905, filed Jan. 5, 1999.

### FIELD OF THE INVENTION

The field of the invention is treatment of retinal and choroidal diseases.

### BACKGROUND OF THE INVENTION

The development of strategies to treat retinal and choroidal diseases is an ongoing therapeutic challenge. Highly specific biologic reagents, which include proteins of relatively high molecular weight, are under development for the treatment of ocular diseases. For example, the overexpression of vascular endothelial growth factor (VEGF) is required for retinal-ischemia associated intraocular neovascularization, leading to proliferative diabetic retinopathy, while mutations in tissue inhibitor of metalloproteinase-3 (TIMP-3) result in Sorsby's macular dystrophy.

Delivery of biologic agents to the retina and choroid is rendered difficult by the fact that the internal limiting membrane (ILM) of the retina is impermeable to linear molecules larger than 40 kDa and globular molecules greater than 70 kDa, precluding intravitreous or topical transcorneal delivery (Smelser et al., In Structure of the eye, II. Rohen E W, ed., Stuttgart: Schattauer-Verlag 109-120, 1965; Peyman and Bok, Invest. Ophthalmol. 11:35-45, 1972; Marmor et al., Exp. Eye Res. 40:687-696, 1985; Misono et al., Invest. Ophthalmol. Vis. Sci. 40(4):S712. Abstract number 3761, 1999) Thus, one of the major problems in the treatment of retinal and choroidal diseases is the delivery of therapeutic levels of medications to target tissues.

Local delivery is the preferred means of achieving therapeutic levels of medications in target eye tissues for two reasons. First, for certain medications, periocular delivery has the potential to increase intraocular concentrations compared to systemic routes by bypassing, for example, the blood-retina barrier (Baum. Int. Ophthalmol. Clin. 13:31, 1973; Baum, Trans. Am. Acad. Ophthalmol. Otolaryngol. 81:151, 1976; Litwack, Arch. Ophthalmol. 82:687, 1969; Weijtens, Amer. J. Ophthalmol. 123:358-63, 1997). Second, while systemic levels are occasionally achieved, local delivery can minimize the side-effects of systemic administration (Weijtens, supra).

Although a variety of local delivery systems for the treatment of posterior segment eye conditions have evolved over the years, each system has limitations. Topical administration is widely utilized in clinical practice but is inefficient for treating posterior segment conditions due to a long diffusional path length, counter-directional intraocular convection, lacrimation, and corneal impermeability to large molecules, and thus requires frequent dosing (Lang, Adv. Drug Delivery Rev. 16:3943, 1995). Depot injections, by either subconjunctival or retro-orbital routes, are a relatively simple and effective means of achieving local concentrations of medications (Baum, 1973, supra; Baum, 1976, supra) but are limited to medications such as antibiotics and corticosteroids and can spill over into the systemic circulation. Intravitreal injection is effective for directed intraocular delivery, but at the same time increases the risk for complications such as vitreous hemorrhage, retinal detachment, and endophthalmitis. Moreover, in chronic conditions, frequent injections are necessary.

Transocular iontophoresis, which uses electrical current to drive ionized drugs into tissues, has been used to deliver antibiotics and corticosteroids into the retina and vitreous (Barza, Opthalmology, 93:133-9, 1997; Lam, Arch. Opthalmol. 107:1368-71, 1989). Yet, transscleral iontophoresis can be accompanied by deleterious retinal necrosis and gliosis, making this method undesirable (Lim, Opthalmology, 100:373-6, 1993).

Other means of drug delivery include biodegradable, controlled-release polymers or liposomal spheres implanted into the vitreous (Brown, J. Pharm. Sci. 72:1181-5, 1983; Kimura, Invest. Ophthalmol. Vis. Sci. 35:28159, 1994; Langer, Nature 263:797-800, 1976; Oritera, Invest. Ophthalmol. Vis. Sci. 32:1785-90, 1991; Peyman, Int. Ophthalmol. 12:175-82, 1988; Tremblay, Invest. Ophthalmol. Vis. Sci. 26:711-18, 1985). Since these polymers and liposomal spheres are placed into the vitreous for intraocular release, these methods have inherent limitations, such as the need for repeated implantation subsequent to drug delivery, and the risk of intraocular injury if the devices are not fixed to the sclera.

Photoactivated liposomes or caged-molecules may hold promise for selective delivery (Asrani et al., Invest Ophthalmol. Vis. Sci. 38:2702-2710, 1997; Arroyo et al., Thromb. Haemost. 78:791-793, 1997); however, radiational and thermal damage associated with these modalities, as well as the limited repertoire of drugs that can be enveloped limit the clinical utility of these approaches at present.

An alternative mode of drug delivery is through the sciera. The large surface area of the sclera compared to the cornea (16.3 cm² vs. 1 cm² in humans) is advantageous since permeability is directly proportional to surface area (Olsen, Am. J. Opthalmol. 125:237-41, 1998). In addition, the sclera has a high degree of hydration, rendering it conducive to water-soluble substances, hypocellularity with an attendant paucity of proteolytic enzymes and protein-binding sites, and there is no significant loss of scleral permeability with age (Olsen, Invest. Opthal. Vis. Sci. 36:1893-1903, 1995).

A variety of factors affect scleral permeability. Age, cryotherapy, and treatment with lasers do not appear to significantly alter human scleral permeability; however, other factors such as surgical thinning are important Surgically thinning the sclera to half its thickness nearly doubles its permeability to a substance (Olsen, 1995, supra).

Previous reports have shown the intraocular passage of a variety of small molecular weight molecules such as penicillins, cephalosporins, gentamicin, amphotericin B, 5-fluorouracil, adriamycin, sulfonamide carbonic anhydrase inhibitors, and ganciclovir (Baum, 1976, supra; Barza, Amer. J. Ophthalmol. 85:541-7, 1978; Edelhauser, Arch. Ophthalmol. 106:1110-5, 1988; Moritera, Invest. Ophthalmol. Vis. Sci. 33:3125-30, 1992; Rubsamen, ARVO abstracts. Invest. Ophthalmol. Visu. Sci. 33:728, 1992; Sakamoto, Arch. Ophthalmol. 113:222-6, 1995; Sanborn, Arch. Ophthalmol. 110-188-95, 1992; Smith, Arch. Ophthalmol. 110:255-58, 1992, Tremblay, Invest. Ophthalmol. Vis. Sci. 26:711-18, 1985).

The transit of higher molecular proteins across the sclera has also been demonstrated. Intraocular injection of albumin (MW 40 kDa) into the suprachoroidal space resulted in its passage out of the eye through the sclera (Bill, Arch. Ophthalmol. 74:248-52, 1965). Similar results were achieved after subconjunctival injection of dextran (MW 70 kDa), albumin (MW 69 kDa), and tissue plasminogen activator (MW 70 kDa) (Lim, Ophthalmol. 100:373-6, 1992; Litwack, Arch Ophthalmol. 82:687, 1969; Maurice, Exp. Eye Res. 25:577-82, 1977; Olsen, 1995, supra).

### SUMMARY OF THE INVENTION

We have developed a minimally invasive transscleral drug delivery modality that can target and unidirectionally deliver therapeutic concentrations of bioactive proteins to the choroid and retina without significant systemic absorption or tissue damage. These methods may be used to treat a number of diseases affecting the retina and choroid.

In a first aspect, the invention features a method for the targeted unidirectional delivery of a therapeutic or diagnostic agent to the eye of a mammal, involving contacting the sclera of the mammal with the therapeutic or diagnostic agent together with means for facilitating the transport of the agent through the sclera.

In a second aspect, the invention features a method for the targeted unidirectional delivery of a therapeutic or diagnostic agent to the eye of a mammal, involving contacting the sclera of the mammal with the therapeutic or diagnostic agent, wherein the agent has a molecular weight of at least 70 kDa.

In one embodiment of the second aspect of the invention, therapeutic or diagnostic agent has a molecular weight of at least 100 kDa. More preferably the therapeutic or diagnostic agent has a molecular weight of at least 120 kDa.

In a third aspect, the invention features a method for the targeted unidirectional delivery of a therapeutic or diagnostic agent to the eye of a mammal, involving contacting the sclera of the mammal with the therapeutic or diagnostic agent, where the agent has a molecular radius of at least 0.5 nm.

In preferred embodiments of the third aspect of the invention, the therapeutic or diagnostic agent has a molecular radius of at least 3.2 nm, or 6.4 nm.

In one embodiment of the above aspects of the invention, prior to contacting the sclera with the agent, the sclera is treated to thin it. Preferably the sclera has a thickness less than 70% of its pre-thinned thickness, and more preferably has a thickness less than 60% of its pre-thinned thickness.

In another aspect of the second or third aspects of the invention, the therapeutic or diagnostic agent is contacted with the sclera together with means for enhancing the transport of the agent through the sclera.

In yet another embodiment of the above aspects of the invention, the device is an osmotic, mechanical, or solid state transport facilitating device, or a polymer. Preferably the device is a pump or comprises microchip.

In still other embodiments of the above aspects the mammal is a human. The method is used to treat a retinal or choroidal disease. In preferred embodiments, the retinal or choroidal disease is selected from the group consisting of macular degeneration, diabetic retinopathy, retinitis pigmentosa and other retinal degenerations, retinal vein occlusions, sickle cell retinopathy, glaucoma, choroidal neovascularization, retinal neovascularization, retinal edema, retinal, ischemia, proliferative vitreoretinopathy, and retinopathy of prematurity.

In further embodiments, the therapeutic agent is selected from the group consisting of purified polypeptides, purified nucleic acid molecules, synthetic organic molecules, and naturally occurring organic molecules. Preferably the polypeptide is an antibody. Most preferably the antibody specifically binds to intercellular adhesion molecule-1.

By a “therapeutic or diagnostic agent” is meant a chemical, be it naturally occurring or artificially-derived, that has a beneficial or diagnostic effect on the eye and can be delivered by transscleral means according to the method of the instant invention. Therapeutic or diagnostic agents may include, for example, polypeptides, synthetic organic molecules, naturally occurring organic molecules, nucleic acid molecules, and components thereof.

As used herein, by “targeted” is meant that a therapeutic or diagnostic agent is delivered only to the sclera.

As used herein, by “unidirectional” is meant that a therapeutic or diagnostic agent is delivered in only one directional, and is therefore delivered to only one site, for example, the sclera.

As used herein, by “facilitating” is meant enhancing the efficacy of the delivery of a diagnostic or therapeutic agent to the sclera.

By “retinal or choroidal disease” is meant a disease or condition in which the retina or choroid function in a diminished capacity as compared to a subject without such a condition, or as compared to the subject itself prior to the onset of the condition or disease. Examples of retinal or choroid diseases include, but are not limited to, macular degeneration, diabetic retinopathy, retinitis pigmentosa and other retinal degenerations, retinal vein occlusions, sickle cell retinopathy, glaucoma, choroidal neovascularization, retinal neovascularization, retinal edema, retinal, ischemia, proliferative vitreoretinopathy, and retinopathy of prematurity.

By “treat” is meant to submit or subject an animal, tissue, cell, lysate or extract derived from a cell tissue, or molecule derived from a cell tissue to a compound in order to lessen the effects of a retinal or choroid disease.

As used herein, by “implant” is meant a device which enhances transport of an agent through the sclera. The implant may be an osmotic, mechanical, or solid state device, or a polymer. Examples of implants include, but are not limited to, pumps with reservoirs containing the desired agent, polymers containing the desired agent, and microchips comprising reservoirs containing the desired agent.

By a “substantially pure polypeptide” is meant a polypeptide that has been separated from the components that naturally accompany it. Typically, the polypeptide is substantially pure when it is at least 60%, by weight, free from the proteins and naturally-occurring organic molecules with which it is naturally associated. Preferably, the polypeptide is at least 75%, more preferably, at least 90%, and most preferably, at least 99%, by weight, pure. A substantially pure serotonin-gated anion channel polypeptide may be obtained, for example, by extraction from a natural source (e.g., a cell derived from ocular tissue) by expression of a recombinant nucleic acid encoding a desired polypeptide, or by chemically synthesizing the protein. Purity can be assayed by any appropriate method, e.g., by column chromatography, polyacrylamide gel electrophoresis, agarose gel electrophoresis, optical density, or HPLC analysis.

A protein is substantially free of naturally associated components when it is separated from those contaminants which accompany it in its natural state. Thus, a protein which is chemically synthesized or produced in a cellular system different from the cell from which it naturally originates will be substantially free from its naturally associated components. Accordingly, substantially pure polypeptides include those derived from eukaryotic organisms but synthesized in E. coli or other prokaryotes.

By a “substantially pure nucleic acid molecule” or “substantially pure DNA” is meant a nucleic acid molecule that is free of the genes which, in the naturally-occurring genome of the organism from which the DNA of the invention is derived, flank the gene. The term therefore includes, for example, a recombinant nucleic acid molecule which is incorporated into a vector, into an autonomously replicating plasmid or virus; or into the genomic DNA of a prokaryote or eukaryote; or which exists as a separate molecule (e.g., a cDNA or a genomic or cDNA fragment produced by PCR or restriction endonuclease digestion) independent of other sequences. It also includes a recombinant nucleic acid molecule which is part of a hybrid gene encoding additional polypeptide sequence.

The present invention provides a means by which to treat macular degeneration, diabetic retinopathy, retinitis pigmentosa, retinal vein occlusions, sickle cell retinopathy, and other diseases of the choroidal and retinal tissues. Defined amounts of the agent can be delivered for prolonged periods of time (weeks to years). The risk of systemic absorption and toxicity is minimal with this method, and intraocular injections, with the concomitant problems of retinal detachment and enthopthalmitis are avoided.

### BRIEF DESCRIPTION OF THE DRAWINGS

FIG. 1A is a least squares regression line of scleral permeability versus molecular radius.

FIG. 1B is a least squares regression line of scleral permeability versus molecular weight.

FIG. 2 is a graph showing scleral effective diffusivities (rabbit, human, and bovine) versus molecular radius (rabbit: diamond; human: square; bovine: maximum value (dark circle), minimum value (light circle)) for various FITC (F−) and rhodamine (R−) dextrans, bovine serum albumin (BSA), radioiodinated human serum albumin (RISA), hemoglobin (Hgb), and inulin. Effective diffusivities were calculated by multiplying permeability coefficients by tissue thickness (0.04 cm for rabbit and 0.06 cm for human). Because of differences in scleral hydration between studies, the data were also converted to yield the effective diffusivity using a mathematical model of transscleral diffusion.

FIG. 3 is a schematic representation of how an osmotic pump may be placed in a rabbit.

FIG. 4 is a graph showing the concentration of FITC-IgG (1 mg/ml delivered at 2.5 μl/h) in the choroid (proximal hemisphere [▪] and distal hemisphere [▴]) and the retina (●). * P<0.01, # P<0.005, † P<.0.001 vs. Day 0. N=4 for all time points.

FIG. 5 is a graph depicting the concentration of FITC-IgG (1 mg/ml delivered at 2.5 μl/h) in the orbit (▪), vitreous humor (▴), and aqueous humor (●). P>0.05 for all tissues at all time points vs. orbital tissue of fellow eye (⋄), which had the highest fluorescence of any tissue in and around that eye. N=4 for all time points.

FIG. 6 is a graph showing the clearance of FITC-IgG (1 mg/ml delivered at 8 μl/h from day 0 to day 1) in the choroid (proximal hemisphere [▪] (t_{1/2}=2.89 d) and distal hemisphere [▴] (t_{1/2}=3.14 d)) and the retina (●) (t_{1/2}=3.36 d). N=4 for all time points.

FIG. 7 is a graph depicting myeloperoxidase (MPO) activity in vitreous humor, choroid, and retina after intravitreous injection of 2 μg VEGF₁₆₅ in eye treated with an anti-ICAM-1 mAb (unshaded bars) or an isotype control mAb (shaded bars). N=5.

### DETAILED DESCRIPTION

As described above, molecules with molecular weights as high as 70 kDa are known to permeate the sclera. Knowledge of the diffusion properties of even larger molecules through sclera is desirable, as several candidate anti-angiogenic drugs are 150 kDa antibodies. The relationship of scleral permeability to molecular weight and molecular (Stokes-Einstein) radius, was determined using an in vitro method of scleral permeability, so that this information may aid in drug development. It has been discovered that large agents can diffuse through thinned sclera, and that biologically relevant concentrations of an agent can be achieved in the retina and choroid via transscleral delivery.

In addition, the results of these studies indicate that unidirectional implants containing therapeutic proteins which are released in a controlled manner and may be used in the treatment of retinal and choroidal diseases. The implant may be an osmotic, mechanical, or solid state device, or a polymer containing the desired diagnostic or therapeutic agent. Examples of such devices include, but are not limited to, osmotic or mechanical pumps, or microchips containing reservoirs of the desired agent, for example in a lyophilyzed form. Such an implant may also have an impermeable backing, for example, plastic to prevent diffusion of the drug into the orbit. If so desired, the implant may contain sufficient therapeutic agent to treat a retinal or choroidal disease for weeks to years.

Using the methods of the invention, immunomodulatory agents and protein-based anti-angiogenic factors may therefore be delivered locally at high concentrations to the retina or choroid. For example, the ability to deliver biological reagents to the choroid and retina in a targeted and minimally invasive fashion can be applied to retinal degenerations such as age-related macular degeneration (ARMD) or retinitis pigmentosa, which may respond to local treatment with VEGF inhibitors or basic fibroblast growth factor, respectively.

Factors such as the rate of release or concentration of the therapeutic or diagnostic agent, the rate of movement of the agent into the target tissue, and the rate of clearance of the agent from the target tissue may all affect the final concentration of therapeutic or diagnostic agents in target tissues. The choice of implant, whether by using an osmotic or mechanical pump, a biodegradable polymer, or some other means, will depend on these factors as well others, such as the length of time that therapy is desired or the size of the diagnostic or therapeutic agent. The advantage of an implant is they allow the release of the agent at a predetermined rate. An additional advantage is that an implant is likely to protect the agent from enzymatic degradation during release.

The techniques described herein may be optimized by determining the best scleral location (equatorial, where the sclera is thinner vs. post-equatorial, where it is thicker), efficacy of scleral thinning by Erbium laser, which will be quantified by ultrasound pachymetry, or lowering intraocular pressure prior to drug delivery, and rate and duration of drug delivery.

The drug delivery methods of the present invention exhibit linear kinetics of absorption and elimination, with the potential to deliver constant doses of medication. These drug delivery methods are robust and are not limited to delivering anti-angiogenic drugs. Such methods may be used to deliver other agents, for example, neuroprotective agents (e.g., fibroblast growth factor or a calcium channel blocker), or any other substantially pure polypeptide known in the art, as well as substantially pure nucleic acid molecules, including vectors for gene transfer, such as DNA plasmids, or viral vectors (e.g., adenoviruses, or adeno-associated viruses). The therapeutic or diagnostic agent to be delivered may also be a synthetic organic molecule, or naturally occurring organic molecule which holds promise in the treatment of glaucoma and other chorioretinal degenerations (Di Polo et al., Proc. Natl. Acad. Sci. USA. 95:3978-3983, 1998; Faktorovich et al., Nature 347:83-86, 1990; Vorwerk et al., Invest. Ophthalmol. Vis. Sci. 37:1618-1624,1996; Bennett et al., Nat. Med. 2:649-654, 1996).

The invention also feature method of delivery a therapeutic or diagnostic agent to the eye of a mammal, where the agent is delivered through sclera which has been treated to thin it, for example, by surgical means.

Animal Models

The use of animals in medical research is an important means to increase our knowledge of the pathogenesis and alleviation of diseases in both animals and humans. Experiments on animals with induced diseases or disorders can be performed under controlled conditions. A successful non-human animal model of retinal or choroidal disease offers the prospect of understanding the origin and mechanisms of these disorders. Existing non-human animal models of retinal or choroidal disorders may also be used, under conditions described herein, to explore potential therapies. Non-human animals may include mice, rats, guinea pigs, hamsters, rabbits, cats, dogs, goats, sheep, cows, monkeys, or other mammals. The use of rabbits in determining pharmacological feasibility is standard practice. Moreover, the scleral permeability of the rabbit is similar to that of bovine and human sclera (Fatt, Exp. Eye Res. 10:243-9, 1970; Maurice, Exp. Eye Res. 25:577-82, 1977; Olsen, 1995, supra). Experiments using human eye bank sclera indicate that the measured permeability to high molecular weight proteins does not differ significantly between rabbit and human sclera.

Animals may be obtained from a variety of commercial sources, for example, Charles River Laboratories, and housed under conditions of controlled environment and diet.

The following examples are to illustrate the invention. They are not meant to limit the invention in any way. Transscleral delivery of agents into the eye, as described in Examples 2-9 below, may be performed with numerous variations. It is understood that variations of the methods described herein may be employed, such variations include, but are not limited to, the variations described below.

### EXAMPLE 1

In Vitro Diffusion of High Molecular Weight Compounds Through Sclera

Isolation and Preparation of Fresh Rabbit Sclera

Dutch-belted rabbits (Pine Acres Rabbitry, Vermont, Mass.), each weighing 2-3 kg, were anesthetized with intramuscular 40 mg/kg ketamine (Abbott, N. Chicago, Ill.) and 10 mg/kg xylazine (Bayer, Shawnee Mission, Kans.). Scleral thickness was measured using a RK-5000 ultrasound pachymeter (KMI Surgical Products, West Chester, Pa.). The eyes were enucleated immediately before sacrifice and immersed in Unisol (Alcon, Ft. Worth, Tex.) for 10 minutes or less. The adherent muscles were excised and episcleral tissue was removed with a sterile gauze sponge. Areas free of nerve and vessel emissaries were used to obtain 7×12 mm slices of sclera under microscopic caliper guidance. Each piece of sclera was used on the day of isolation.

In Vitro Diffusion Apparatus

A 5×10 mm window, 2 mm from the bottom, was created on one face of a spectrophotometry polystyrene cuvette (Sigma, St. Louis, Mo.) using a Bridgeport vertical milling machine (Bridgeport, Bridgeport, Conn.), and a piece of sclera was blotted dry and placed over this window without stretching so as not to induce asymmetric stresses. A small amount of cyanoacrylate tissue adhesive (Ellman International, Hewlett, N.Y.) was applied to the entire boundary of the tissue rim to seal its cut surface to the cuvette and prevent leakage around the sclera, and a second identical cuvette was aligned with the first cuvette and glued in place over the tissue. After the glue polymerized, within 3 to 4 minutes, the cuvette facing the “orbital” surface of the sclera was filled with Unisol. The apparatus was discarded if leakage into the “uveal” chamber was observed. Unisol was replaced with diffusion medium (see below) and the apparatus was incubated at 37° C. in 5% CO₂ atmosphere for 1 hour to restore normal hydration and temperature.

Diffusion Medium

Hanks' balanced salt solution without phenol red, containing 1% glutamine-penicillin and streptomycin, tetracycline (48 μg/ml), and aprotinin (1.5 μg/ml) (all from Sigma), was used as the diffusion medium. Tetracycline and aprotinin were excluded from the medium for certain experiments with FITC-BSA to evaluate the effect of these proteolysis inhibitors on scleral permeability. The pH of all solutions ranged from 7.41 to 7.45.

Fluorescent Compounds

Fluorescein isothiocyanate (FITC) conjugated dextrans ranging in molecular weight from 4 kDa to 150 kDa, FITC-bovine serum albumin (BSA), FITC-rabbit IgG (all from Sigma), rhodamine-conjugated dextran of molecular weight 70 kDa (Molecular Probes, Eugene, Oreg.), and sodium fluorescein (Akorn, Abita Springs, La.) were studied. At least 5 experiments were performed on each compound. To confirm that the parent compound was not cleaved from FITC, selected samples were subjected to protein precipitation with 20% trichloroacetic acid (Pohl and Deutscher, Guide to Protein Purification. San Diego: Academic Press; 68-83, 1990). Samples were protected from light at all times before fluorescence measurements.

Sample Collection

The medium in each “uveal” chamber was replaced by 4 ml of fresh medium at 37° C., while the “orbital” chamber was filled with an equal volume of diffusion medium containing 1 mg/ml of a fluorescent compound, freshly prepared and warmed to 37° C. Experiments were performed in a tissue culture incubator at 37° C. in a 5% CO₂ atmosphere. Samples measuring 0.4 ml were removed from each chamber at 30 minute intervals for 4 hours and stored at −80° C. Solutions were stirred before each sample collection.

Scleral Hydration

The water content of sclera was measured by comparing the wet weight of freshly obtained tissue to its dry weight, obtained by subjecting the tissue to drying at 100° C. for 3 hours. The effect of the diffusion medium on scleral hydration ([Wet weight−Dry weight]/Wet Weight) was examined by comparing the water content of sclera exposed to the experimental apparatus for 4 hours to fresh sclera.

Scleral Permeability Coefficient

Diffusion from the “orbital” chamber to the “uveal” chamber was characterized by means of a permeability coefficient (P_{c}), which is the ratio of steady-state flux (the mass of solute crossing a planar unit surface normal to the direction of transport per unit time) to the concentration gradient (Burnette, Theory of mass transfer. In: Controlled Drug Delivery. 2nd ed. Vol. 29. Robinson J R, Lee VHL, eds., New York: Marcel Dekker, 95-138, 1987). In these experiments, the concentration in the “uveal” chamber, Cᵤ, was a negligible fraction of the concentration in the “orbital” chamber, Cₒ, which did not change measurably over the course of the experiment. Within 30 minutes steady state diffusion was achieved; therefore, the permeability coefficient was calculated as follows:

$${P}_{c} = \frac{( \overline{{C}_{u4}} - \overline{{C}_{u0 .5}} ) {V}^{*}}{{AtC}_{o}}$$

where \overline{C_{u0.5}} and \overline{Cᵤ₄} are the concentrations in the “uveal” chamber at 0.5 and 4 hours, respectively, estimated by linear regression on the concentration of the 8 collected samples, V* is the corrected chamber volume (4 ml divided by 3.6, to correct for the volume changes induced by sampling), A is the surface area of exposed sclera (0.84 cm²), and t is duration of steady state flux (3.5 hours).
Analysis of Scleral Integrity

At the conclusion of selected experiments, the diffusion apparatus was thoroughly cleansed with Unisol, and the permeation of sodium fluorescein was observed and compared with diffusion kinetics of sodium fluorescein across fresh sclera to unmask possible damage to the sclera. The effect of cyanoacrylate tissue adhesive on scleral ultrastructure was examined by transmission electron microscopy.

Fluorescence Measurements

Fluorescence was measured at room temperature (25° C.) with a MPF-44A fluorescence spectrophotometer (Perkin-Elmer, Newton Center, Mass.) in a right-angle geometry. For FITC-compounds, excitation and emission wavelengths were 492.5 nm and 520 nm, respectively. For rhodamine conjugated dextran, excitation and emission wavelengths were 570 nm and 590 nm, respectively. Standard curves of fluorescence versus concentration were obtained by serial dilution of fluorescent compounds in diffusion medium. Concentrations in samples were determined by linear regression analysis within the linear portion of the standard curve.

Statistics

Unpaired Student's t-test was used to compare continuous variables. All P values were two-tailed. An α level of 0.05 was used as the criterion to reject the null hypothesis of equality of means.

Results

After the first 30 minutes of each experiment there was a constant flux of the fluorescent compound across the sclera. The permeability of sclera to the tracers studied is shown in Table 1.

TABLE 1
Permeability of Sclera to Tracers of Varying Molecular Weight and
Molecular Radius

| | | | Permeability |
|---|---|---|---|
| | | | coefficient |
| | Molecular | Molecular | (×10⁻⁶ cm/s) |
| Tracer | weight (D) | radius (nm) | (mean ± sd) |
| Sodium fluorescein | 376 | 0.5 | 84.5 ± 16.1 |
| FITC-D-4 kDa | 4,400 | 1.3 | 25.2 ± 5.1 |
| FITC-D-20 kDa | 19,600 | 3.2 | 6.79 ± 4.18 |
| FITC-D-40 kDa | 38,900 | 4.5 | 2.79 ± 1.58 |
| FITC-BSA | 67,000 | 3.62 | 5.49 ± 2.12 |
| Rhodamine D-70 kDa | 70,000 | 6.4 | 1.35 ± 0.77 |
| FITC-D-70 kDa | 71,200 | 6.4 | 1.39 ± 0.88 |
| FITC-IgG | 150,000 | 5.23 | 4.61 ± 2.17 |
| FITC-D-150 kDa | 150,000 | 8.25 | 1.34 ± 0.88 |

The molecular (Stokes-Einstein) radii were culled from the literature (Jain, Biotechnol. Prag. 1: 81-94, 1985; Nugent and Jain, Am. J Physiol. 246: H129-137, 1984; Potschka, Anal. Biochem. 162: 47-64, 1987; Prausnitz and Noonan, J. Pharm. Sci.; 87: 1479-1488, 1998).
FITC = Fluorescein isothiocyanate,
D = dextran.

Sodium fluorescein, the smallest compound, had the highest permeability coefficient (84.5±16.1×10⁻⁶ cm/s), whereas FITC dextran 150 kDa, which had the largest molecular radius, had the lowest permeability coefficient (1.34±0.88×10⁻⁶ cm/s). The permeability coefficients of rhodamine conjugated dextran 70,000 D and FITC-dextran 71,200 D were not significantly different (P=0.88), strengthening the reliability of the paradigm. The sclera was more permeable to the two proteins tested (BSA and IgG) than to dextrans of comparable molecular weight.

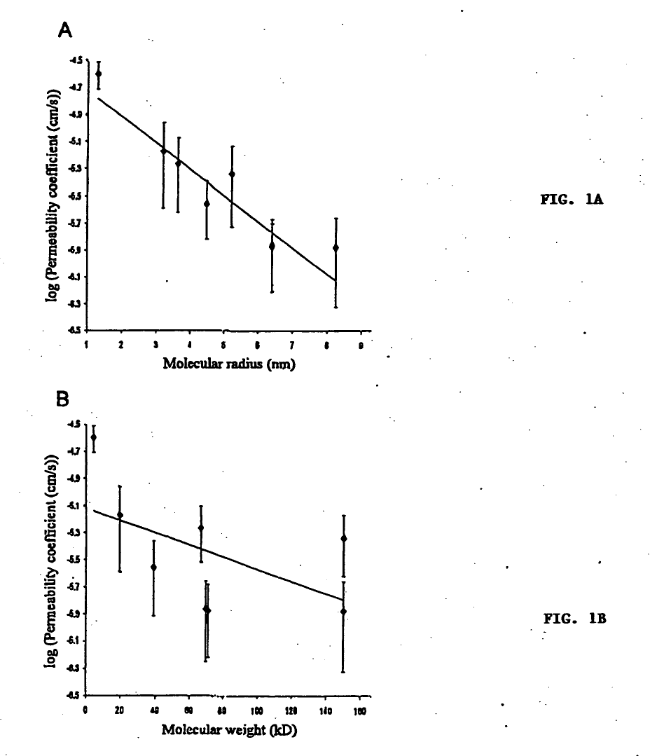

Scleral permeability declined exponentially with increasing molecular weight and molecular radius. Log-linear regression analysis demonstrated that molecular radius was a better predictor of permeability (r²=0.87, P=0.001) than molecular weight (r²=0.31, P=0.16) (FIGS. 1A and 1B).

Random samples containing FITC-BSA and FITC-IgG were subjected to protein precipitation with trichloroacetic acid following diffusion through sclera. The fluorescence of the resulting supernatants was not different from that of the diffusion medium, indicating there was no significant dissociation of the FITC conjugate.

The permeability coefficient of sodium fluorescein across fresh sclera (84.5±16.1×10⁻⁶ cm/s) was not significantly different from that across sclera previously used in a 4-hour in vitro diffusion apparatus (76.3±24.1×10⁻⁶ cm/s) (P=0.55). Transmission electron microscopy of sclera exposed to cyanoacrylate tissue adhesive demonstrated normal collagen fibrils in closely packed lamellae as well as normal banding patterns and fibril diameters of collagen throughout the scleral stroma. In addition, there was no demonstrable difference between cyanacrylate exposed and control sections, either in density of packing or in maximal width of individual collagen fibrils. There was no difference between tissue hydration of fresh sclera (69.5%±0.9%) versus sclera exposed to diffusion medium for 4 hours (69.2%±0.4%) (P=0.66). Mean scleral thickness was 416±21 μm.

In sum, these data indicate that the sclera is quite permeable to high molecular weight compounds. In an ideal aqueous medium the Stokes-Einstein equation predicts that permeability declines as a linear function of molecular radius. However, in porous diffusion through a fiber matrix such as the sclera, permeability declines roughly exponentially with molecular radius, as observed in these experiments (Edwards, Am. Inst. Chem. Eng. J; 44:214-225, 1998; Cooper and Kasting, J. Controlled Release; 6:23-35, 1987). The information obtained through these studies can be used to design therapeutics which are more likely to permeate normal or thinned sclera.

For all molecules studied, constant flux of compounds across the sclera occurred by 30 minutes, similar to observations in human sclera (Olsen, 1995, supra). This is consistent with documented stability of FITC conjugation to the parent compound and the absence of aggregation (Schröder U et al., Microvasc. Res. 11:33-39, 1976). In addition, protein precipitation revealed that the proteins studied, BSA and IgG, remained intact as they diffused across the sclera. The similarity in permeability coefficients of rhodamine dextran 70,000 D and FITC dextran 71,200 D reinforce the fidelity of the experimental design.

The use of proteolysis inhibitors (aprotinin and tetracycline) to limit tissue degradation and simulate in vivo sclera, which has a paucity of proteolytic enzymes in the absence of inflammation or injury, did not alter scleral permeability (Foster and Sainz de la Maza M, The Sclera. New York: Springer-Verlag; 1994). For example, the scleral permeability to FITC-BSA in media with proteolysis inhibitors with (5.49±2.12×10⁻⁶ cm/s) and without proteolysis inhibitors (5.21±1.85×10⁻⁶ cm/s) (P=0.89) was not significantly different.

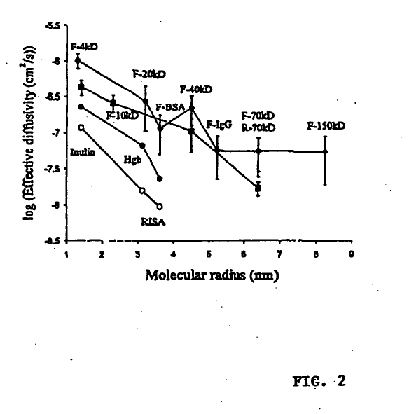

To better understand how the results of these in vitro studies translate into therapies in humans and other animals, the results can be compared to the reported permeability of human and bovine sclera (Olsen, 1995, supra; Maurice, supra) (FIG. 2). The permeability coefficients for rabbit and human sclera were converted to effective diffusivities (which are thickness invariant) by assuming a thickness of 0.04 cm and 0.06 cm, respectively, and accounting for variations in scleral hydration using computer simulation of a mathematical model of transscleral diffusion (Edwards and Prausnitz, supra).

### EXAMPLE 2

Osmotic Pump Implantation

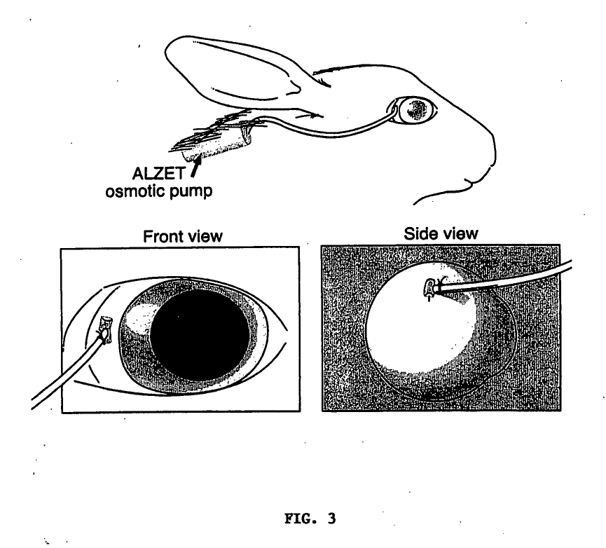

Dutch-belted rabbits were anesthetized with intramuscular ketamine (40 mg/kg; Abbott, N. Chicago, Ill.) and xylazine (10 mg/kg; Bayer, Shawnee Mission, Kans.). Osmotic pumps (ALZET, ALZA, Palo Alto, Calif.) were loaded with drug and incubated at 37° C. prior to implantation. The osmotic pump was implanted subcutaneously between the scapulae and connected to a brain infusion kit (ALZA), which was modified so that the tip could be secured to, and face, the orbital surface of the sclera with a single biodegradable polyglactin 910 suture (Ethicon, Somerville, N.J.) in the superotemporal quadrant of the eye, 14 to 16 mm posterior to the limbus (near the equator) (FIG. 3). Care was taken to make a partial thickness pass through the sclera. If uvea, blood or vitreous was observed during the procedure, the experiment was terminated.

### EXAMPLE 3

Collection of Ocular Tissue and Blood

Blood was collected by cardiac puncture prior to surgical enucleation of the eyes under deep anesthesia. Aqueous humor of each eye was collected using a 30-gauge needle. Vitreous humor, retina, choroid, and orbital tissue of both eyes were dissected and isolated under a microscope. The choroid of the treated eye was separated into two hemispheres, proximal (in which the tip of the pump was centered) and distal to the tip of the pump. Animals were sacrificed with intracardiac pentobarbital (100 mg/kg) (Vortech, Dearborn, Mich.).

### EXAMPLE 4

Fluorescence Measurements

A Perkin-Elmer Fluorescence spectrophotometer, model MPF-44A (Perkin Elmer Corporation, Newton, Mass.) was used to determine specimen fluorescence. Excitation and emission wavelengths were set at 465 nm and 525 nm, respectively, in a right angle geometry using 3 nm band widths. Variables affecting the performance of the spectrophotometer, such as fluctuations of the excitation power, gain of the photomultiplier, and the spectral sensitivity of the instrument, were adjusted.

### EXAMPLE 5

Transscleral Delivery of Immunoglobulins

ALZET 2ML4 osmotic pumps (4 weeks, 2.5 μl/h) containing fluorescein isothiocyanate conjugated (FITC) rabbit IgG (15.5 mg/ml) (Sigma, St. Louis, Mo.) were implanted in one eye of each animal. Animals were sacrificed at 3, 5, 13,20, and 28 days after implantation, and fluorescence was measured in ocular tissues and plasma. Clearance of FITC-IgG was determined by implanting ALZET 2001D osmotic pumps (24 h, 8 μl/h) in one eye of each animal for 1 day, and measuring fluorescence in ocular tissues at 1, 3, 5, and 9 days after explantation.

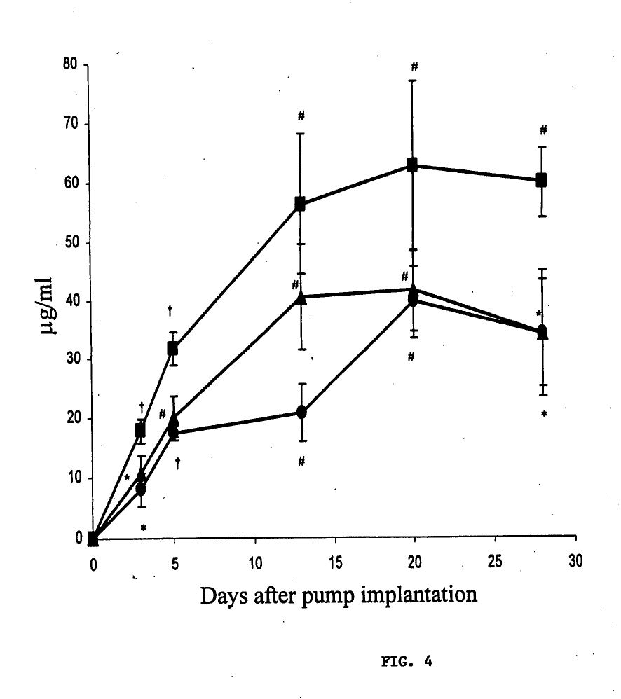

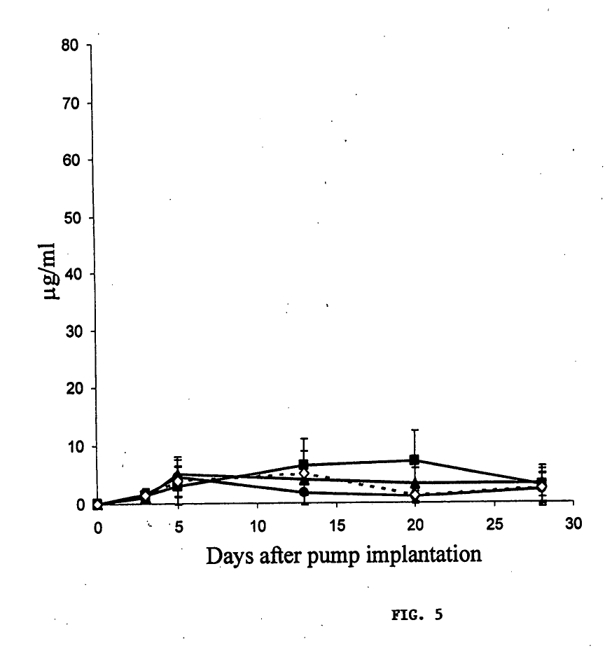

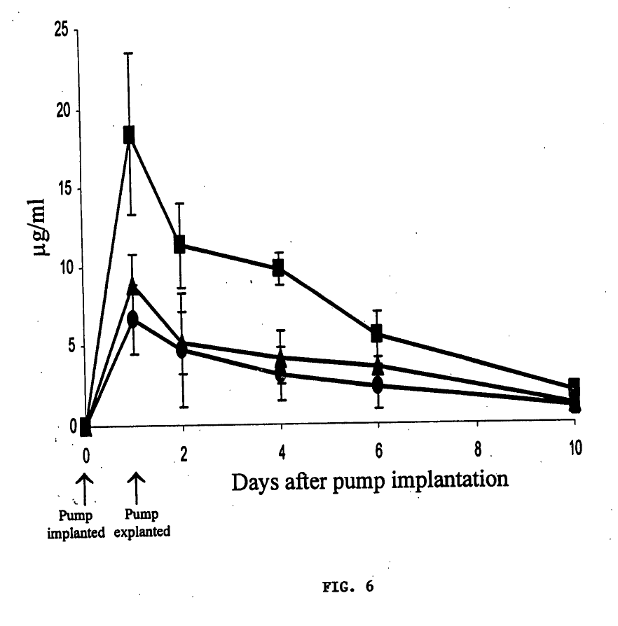

The following analyses were done following scleral implantation of the osmotic pump. FITC-IgG was delivered to the superotemporal scleral surface at a rate of 2.5 μl/h for 28 days via an osmotic pump. Levels of retinal and choroidal fluorescence, a quantitative marker of IgG concentration, were significantly higher than baseline at all time points (FIG. 4) (n=4 per time point, P≦0.01 for each time point). Levels in the orbit, vitreous humor, and aqueous humor were negligible (FIG. 5) (n=4 per time point, P>0.05 for each time point). No fluorescence was detected in the plasma at any time point. The concentration of IgG in the choroid in the hemisphere proximal to the pump, which reached a plateau of 6% of the concentration in the osmotic pump, was roughly 50% greater than in the distal hemisphere, and 50% greater than the overall retinal concentration. The elimination of fluorescence from the choroid and retina followed first-order kinetics with half-lives of approximately 3 days (FIG. 6) (n=4 per time point).

To confirm the continued linkage of FITC to IgG, protein precipitation of tissue homogenates at various time points was performed. Virtually all fluorescence was protein-bound (99.6% in retina and 99.8% in choroid, at 28 days (n=3)), indicating that the IgG molecule crossed the sclera intact and did not undergo significant cleavage over the time studied. Additionally, the in vitro transscleral diffusion of fluorescence from retinal tissue homogenates (mean permeability coefficient=6.2×10⁻⁶ cm/s) and choroidal homogenates (mean permeability coefficient=5.6×10⁻⁶ cm/s) was not significantly different (P>0.05 for both comparisons) from that of FITC-IgG (mean permeability coefficient=4.6×10⁻⁶ cm/s), indicating that the tissue fluorescence emanated from intact FITC-IgG (Ambati et al., Invest. Ophthalmol. Vis. Sci, in press). Iatrogenic perforation of the sclera at the injection site did not result in increased intraocular delivery (Table 2), indicating that lateral surface diffusion did not play a significant role in transscleral entry.

TABLE 2
Effect of Iatrogenic Perforation of the Sclera on Intraocular Delivery of
FITC-IgG

| | Without scleral | With scleral | |
|---|---|---|---|
| Tissue | perforation | perforation | P |
| Choroid, proximal | 1.84 ± 0.51% | 2.06 ± 0.36% | 0.67 |
| hemisphere | | | |
| Choroid, distal | 0.88 ± 0.20% | 0.99 ± 0.14% | 0.58 |
| hemisphere | | | |
| Retina | 0.66 ± 0.22% | 0.55 ± 0.08% | 0.60 |
| Vitreous humor | 0.04 ± 0.06% | 0.12 ± 0.04% | 0.19 |

Concentration of FITC-IgG (delivered for 24 h at 8 μl/h) in tissues as a percentage of its concentration in osmotic pump, with and without the presence of a scleral perforation in the inferonasal pars plana with a 30-gauge needle.

### EXAMPLE 6

Analysis of Possible Enzymatic Degradation of FITC-IgG

Choroid and retina obtained from eyes in which osmotic pumps containing FITC-IgG were implanted were subjected to protein precipitation with 20% trichloroacetic acid (Sigma; Pohl, supra) and the supernatants were assayed for residual fluorescence, which would suggest cleavage of FITC from IgG. As a confirmatory test, tissue homogenates were placed in a diffusion chamber separated by fresh virgin sclera to determine diffusion kinetics of fluorescent molecules in the tissue, which was compared to diffusion kinetics of FITC-IgG, as significant differences between the diffusion of tissue homogenate fluorescence and the diffusion of native FITC-IgG also would suggest cleavage of FITC from IgG (Ambati, Supra). Briefly, and as described in full detail in Example 1, an in vitro transscleral diffusion apparatus was constructed by attaching fresh sclera to 2 spectrophotometry polystyrene cuvettes (Sigma), each with a 5×10 mm window fashioned 2 mm from the bottom, with a small amount of cyanoacrylate tissue adhesive (Ellman International, Hewlett, N.Y.). Transscleral diffusion of fluorescent molecules at 37° C. in a 5% CO₂ atmosphere was determined by sampling every 30 minutes over 3 hours.

### EXAMPLE 7

Scleral Thinning

Thinning of the sclera was carried out using a surgical technique in a rabbit model. Two Dutch belted rabbits (Pine Acres Rabbitry, Vermont, Mass.), weighing three kilograms each, were anesthetized with intramuscular injections of a mixture of 40 mg/kg ketamine (Ketalar, Parke-Davis, Morris Plains, N.J.) and 10 mg/kg Xylazine (Bayer, Shawnee Mission, Kans.). Proparacaine hydrochloride (0.5%) topical anesthetic drops (Alcon, Humancao, Puerto Rico) were administered before placement of lid speculae. Osmotic pumps were secured to the sclera with sutures after lamellar scleral resection.

A 360° conjunctival peritomy, followed by identification and isolation of the recti muscles, was performed. A suitable location was identified in the superotemporal quadrant and a partial thickness sclerotomy was performed according to standard procedures. The resulting scleral pocket, measuring 0.5 mm×2.0 mm involving 50% scleral thickness, was created in a vertical fashion 5.5 mm from the limbus using a Beaver blade (Grieshaber, Schaffhausen, Germany).

### EXAMPLE 8

Delivery of Immunoglobulins Through Thinned Sclera

A purified rabbit immunoglobulin G conjugated to fluorescein isothiocyanate (FITC-IgG) (Product No. F-7250, Sigma Chemical Company, St. Louis, Mo.), with a molecular weight of 150 kDa, was used as the testing compound. Alternatively, Pacific Blue-conjugated IgG may also be used (Molecular Probes, Eugene, Oreg.). The solution contained 15.4 mg protein/ml and had a F/C molar ratio of 5.0. FITC is not cleaved from the parent compound, after diffusion through the sclera, as measured by protein precipitation using 20% trichloroacetic acid (Sigma, St. Louis, Mo.). That the fluorescent measurements of the tissues are those of intact FITC-IgG may also be demonstrated by SDS-PAGE followed by fluorometry. The fluorescent material was protected from light to prevent degradation.

A unidirectional osmotic minipump may be used to deliver the FITC-IgG at a fixed rate to the orbital scleral surface of locally anesthetized rabbits. The minipump (Alzet 2001D, ALZA Corporation, Palo Alto, Calif.), which contained a 200 μl reservoir, was retrofitted using 40 mm of silicone tubing to a infusion cannula with a 4 mm metallic tip (Alzet Brain Infusion Kit, ALZA Corporation, Palo Alto, Calif.) in order to direct microperfusion of the immunoglobulin solution over a limited area into the target tissue. The osmotic minipump, which was tested for delivery of immunoglobulins, infuses solutions at a mean pump rate of 8.25 μl per hour.

The minipump reservoir was filled according to instructions for operation from the manufacturer. The flow moderator was removed and the reservoir filled with undiluted FITC-IgG using a 25 gauge needle attached to a 1 cc syringe. The filled pump weight was determined and the pump placed in 0.9% saline at 37° C. for at least four hours to equilibrate the device. Prior to placement, the infusion tubing was checked for functional delivery of the immunoglobulin solution.

A surgical procedure was used to implant the infusion tubing of the osmotic minipump. Rabbits were prepared as described in Example 7. The scleral pocket accommodated the metallic infusion port, which was then sutured into place using interrupted 8-0 Nylon sutures (Ethicon, Somerville, N.J.). The infusion port was connected to a flexible tubing which was fixed to the sclera using 6-0 Vicryl (Ethicon, Somerville, N.J.) sutures. The tube was connected to the osmotic pump, which was fixed extraorbitally on top of the head using tape, as the limited orbital volume prevented intraorbital placement of the reservoir. The conjunctiva was reapproximated over the tubing using 6-0 Vicryl sutures.

The rabbits were sacrificed at 6 hours and 24 hours after surgical placement of the osmotic pumps. The pump and cannula were removed and the empty pumps weighed. Both eyes were enucleated immediately prior to euthanasia, and individual tissues isolated with the aid of an operating microscope. The amount of drug in the tissues was quantified by fluorometry. The contralateral eye, which did not have an osmotic pump, served as a control. Maximal amounts of aqueous fluid and vitreous humor were obtained prior to opening the globe. The globe was opened using a razor blade and splayed as a single specimen before intraocular tissue such as retina and choroid were stripped in their entirety. Representative samples of other solid tissues, including orbital fat and sclera, were also harvested. Blood was sampled from an ear vein prior to the experiment (t=0), and at 6 hour (t=6) and 24 hour (t=24) timepoints. The final blood sample was obtained by intracardiac puncture before delivery of a lethal dose of anesthetic. All specimens were frozen at −80° C. prior to spectrophotometry. Solid retina and choroid specimens were disrupted using a tissue sonicator for five minutes. The tissues were diluted in 300 μl of normal balanced saline. The blood specimens were centrifuged and the supernatant extracted for measurement.

The following analyses were performed to determine the efficacy of thinned transscleral IgG delivery. Determination of antibody concentrations in the blood is important as this can confound the final intraocular concentrations. To determine if the intraocular results could be accounted for by systemic delivery, the serum levels of FITC-antibody was assayed. Such levels were measured as described in Example 4. Minimal levels of detection of blood samples at 6 hours and 24 hours support that systemic absorption of FITC-antibody provide a negligible contribution to intraocular concentrations. Such results confirm previous reports of rabbits dosed with medication in one eye, which achieve negligibly low levels in the non-treated eye (Ahmed, Invest. Ophthalmol. Vis. Sci. 26:584-87, 1985).

Table 3 shows the results of transscleral delivery of either FITC labeled IgG using osmotic pumps implanted subcutaneously on the backs of female Dutch-belted rabbits and connected to a brain infusion kit (BIK) carrying the IgG to the eye. In all animals, the distal end of the BIK was 12 to 16 mm posterior to the limbus in the inferotemporal quadrant. In animals 1 and 6, the BIK was sutured to the scleral surface; in animals 2 to 5, a scleral flap was raised and the BIK tip was placed under the scleral flap, which was then closed.

Values for each tissue were corrected for the background auto-fluorescence of the tissue values from the non-implanted fellow eye. The choroid was collected as two hemispheres (nearer to or farther from the pump tip). The autofluorescence of choroid and retina from fellow eyes was not significantly difference from that of tissue from animals which were not exposed to dye, and the fluorescence per gram weight of tissue was virtually constant (standard error of the mean was less than 10%).

TABLE 3
Transscleral Delivery of IgG in Rabbits. Percentage of
Drug Delivered (grams drug/grams tissue or grams
drug/ml tissue).
Time (hrs)

| | 15 | 15 | 18 | 24 | 24 | 24 |
|---|---|---|---|---|---|---|
| Animals | 1 | 2 | 3 | 4 | 5 | 6 |
| AC, Vitreous | 0 | 0 | 0 | 0 | 0 | 0 |
| Retina | 1.0 | 1.6 | 1.9 | 2.7 | 2.0 | 0.9 |
| Near Choroid | 1.9 | 4.7 | 5.5 | 6.5 | 6.1 | 2.1 |
| Far Choroid | 0.8 | 1.5 | 1.7 | 2.7 | 1.9 | 0.8 |

AC = anterior chamber

The results indicate that 5.1±2.1% of the agent can be delivered transsclerally to the near choroid; 1.8±1.0% can be delivered to the far choroid; and 1.3±1.0% can be delivered to the retina. The effect of modulating intraocular pressure or altering scleral thickness by erbium YAG laser surgery on the spatiotemporal characteristics of transscleral flux may be determined by fluorometry.

### EXAMPLE 9

Transscleral Delivery of Anti-Angiogenic Drugs

Antibodies that Bind VEGF

Age-related macular degeneration (ARMD) is the leading cause of blindness among the elderly in the developed world and affects some 15 million people in the United States alone. The neovascular form of ARMD, characterized by choroidal neovascularization (CNV), accounts for 80% of the visual loss in these patients. A compelling body of evidence suggests that vascular endothelial growth factor (VEGF), a 46 kDa homodimeric globular glycoprotein, is operative in the development of CNV. Systemic delivery of anti-VEGF antibodies may not achieve sufficient intraocular levels. Furthermore, it may undesirably inhibit the physiological function of VEGF in such organs as the heart, limbs and reproductive systems (Ergun, 13:19-20, 1997; Ku, Am. J. Physiol. 265:H586-92, 1993; Takeshita, Am. J. Pathol. 147:1649-60, 1995; Torry, Fertility and Sterility 66:72-80, 1996).

Transscleral delivery of antibodies that bind VEGF avoids both the above-mentioned problems. The efficacy of transscleral delivery of anti-angiogenic drugs in preventing CNV may be tested using a monkey model of experimentally induced choroidal neovascularization. The distinct retinal and choroidal circulation and macular anatomy in the monkey are similar to those of humans.

CNV is created by placing high intensity argon laser burns in the maculae of cynomolgus monkeys (Macaca fascicularis). Angiographically documented CNV typically develops 2 or 3 weeks (mean of 2.9 weeks) after laser treatment in 39% of the lesions, with increased expression of VEGF seen as early as 1 week after laser treatment (Ohkuma, Arch. Opthalmol. 101:1102, 1983; Ryan, Arch. Opthalmol. 100:1804, 1982). By placing 7 lesions in each macula, greater than 90% of eyes develop CNV.

The animals are anesthetized for all procedures with intramuscular injections (IM) of a mixture of ketamine, 20 mg/kg; diazepam, 1 mg/kg (Elkins-Sinn Inc., Cherry Hill, N.J.); and atropine sulfate, 0.125 mg/kg (Gensia Laboratories Ltd., Irvine, Calif.). Supplemental anesthesia of ketamine (10 mg/kg IM) assures stable anesthesia. Proparacaine hydrochloride (0.5%) topical anesthetic drops are administered before placement of any lid speculae and for pneumotonometry. Pupils are dilated as needed with 2.5% phenylephrine and 0.8% tropicamide drops. Animals are placed in a comfortable restraint device to allow head positioning for photography and angiography. Intravenous medications are administered using IV tubing, sterile IV 24 gauge catheters and a pediatric infusion pump (IVAC 710 syringe pump).

The animals will undergo baseline fundus photography and fluorescein angiography. On day 0 both eyes will undergo argon green laser (514 nm) treatment to induce CNV. Seven laser burns (50 μm spot size, 0.1 seconds, 350-450 MW) will be placed in each macula. Immediately after laser treatment one eye of each animal will be randomized to receive anti-VEGF antibody as per the optimized transscleral delivery mode determined above. The fellow eye with receive an equimolar amount of an isotype control drug or the vehicle alone. The animals will be followed weekly with biomicroscopy, color fundus photography and fluorescein angiography to 4 weeks. Angiograms will be graded in masked fashion using the standardized grading system developed for this model. Because the contribution of uveoscleral outflow to total outflow is higher in cynomolgus monkeys than in rabbits (Nilsson, Eye 11:149-154, 1997), the effect of intraocular pressure modulation on achieving sufficient intraocular levels of anti-angiogenic drug will be assessed. Deeply anesthetized animals will be sacrificed immediately following harvest of eyes, Freshly enucleated eyes will be fixed in 4% paraformaldehype, embedded in paraffin and sectioned. Histopathological comparison of treated and untreated eyes will be performed through morphometric analysis of serial sections.

The volume of the choroid in the monkey is approximately 0.2 to 0.25 ml. A steady state concentration of 1 μg/ml anti-VEGF antibody is approximated to be the minimum required to inhibit CNV development. Assuming that 1% of anti-VEGF can be delivered transsclerally per ml of choroid, 30 mg of drug will be sufficient for a year.

Antibodies that Bind ICAM-1

VEGF induces the expression of intercellular adhesion molecule-1 (ICAM-1) in tumor and retinal vascular endothelium, and regulates leukocyte adhesion to endothelial cells (Melder et al., Nat Med. 2:992-997, 1996; Lu et al., Invest. Ophthalmol. Vis. Sci. 40:1808-1812, 1999). Inhibition of ICAM-1 also decreases VEGF-induced leukostasis and angiogenesis in the cornea (Becker et al., Invest. Ophthalmol. Vis. Sci. 40:612-618, 1999). As ICAM-1 mediates leukocyte endothelial adhesion and extravasation into surrounding tissue, myeloperoxidase (MPO) activity can be used to quantify the tissue sequestration of leukocytes (Makgoba et al., Nature. 331:86-88. 31, 1988; Bradley et al., J. Invest. Dermatol. 78:206-209, 1982). The transscleral delivery of a mouse anti-human ICAM-1 monoclonal antibody, which inhibits rabbit neutrophil adhesion through cross-reactivity to rabbit ICAM-1, was investigated to determine if it could inhibit VEGF-induced leukostasis in the choroid and retina by measuring MPO activity in these tissues.

ALZET 2001D osmotic pumps, one containing mouse anti-ICAM-1 IgG2a mAb (2 mg/ml) from clone BIRR0001 (Robert Rothein, Boehringer Ingelheim, Ridgefield, Conn.), and one containing mouse non-immune IgG2a mAb (2 mg/ml; R&D Systems, Minneapolis, Minn.) were implanted in the superotemporal quadrant of each eye. The surgeon was masked to the identity of the two pumps. Six hours after implantation, animals were anesthetized, and 0.5% proparacaine (Alcon, Ft. Worth, Tex.) and 0.3% ofloxacin (Allergan, Hormigueros, P R) eye drops were topically applied. Following pump placement, 2 μg of human recombinant vascular endothelial growth factor (VEGF₁₆₅) (Napoleone Ferrara, Genentech, San Francisco, Calif.), diluted in 100 μl of pyrogen-free Dulbecco's phosphate buffered saline (PBS) (Sigma), was injected into the vitreous body through the inferonasal pars plana of each eye with a 30-gauge needle. To normalize intraocular pressure, 100 μl of aqueous humor was removed with a 30-gauge needle. Animals were sacrificed 24 hours after implantation and myeloperoxidase activity was measured in ocular tissues. To ensure that the intravitreous injection did not provide an intraocular conduit for the antibodies, 2 animals were implanted with ALZET 2001D osmotic pumps containing FITC-IgG and a 30-gauge needle was used to perforate the inferonasal sclera. The fluorescence in ocular tissues, 24 hours later, was compared to that in animals without the perforation.

Myeloperoxidase Assay

Myeloperoxidase (MPO) was extracted by freezing, thawing, and sonicating tissue in 50 mM potassium phosphate buffer, pH 6.0 (Sigma) containing 0.5% hexadecyltrimethylammonium bromide (Sigma) three times. MPO activity in supernatants was measured by the change in absorbance at 460 nm resulting from decomposition of 0.0005% hydrogen peroxide in the presence of 0.167 mg/ml O-dianisidine (Sigma) (Bradley, supra), and compared to the activity of 1 unit of MPO (Sigma), using a MR4000 microplate reader (Dynatech, Chantilly, Va.). The assay was performed in masked fashion.

Bioactivity of Transsclerally Delivered Protein

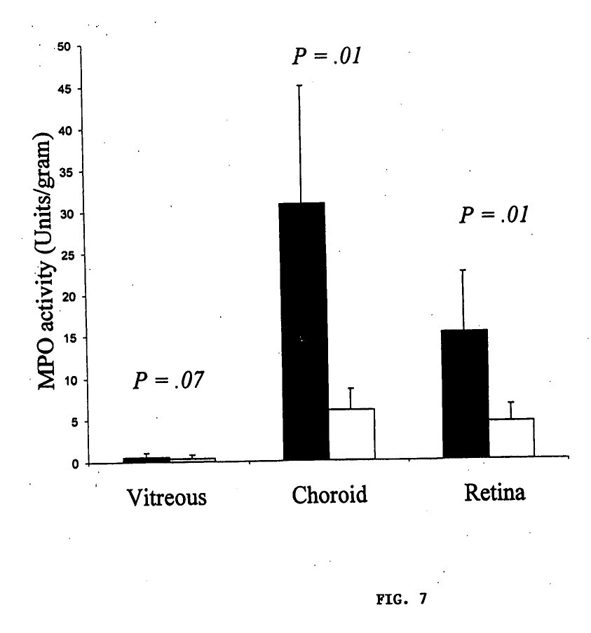

VEGF-induced leukostasis in the retina and choroid, as measured by myeloperoxidase (MPO) activity, was markedly inhibited by the delivery of anti-ICAM-1 mAb (FIG. 7). MPO activity in the choroid of the eye treated with anti-ICAM-1 mAb (2 mg/ml delivered at 8 (μl/h) was 80% less (P=0.01) than in the eye receiving an equal rate of delivery of an isotype control antibody (n=5). Inhibition of MPO activity in the retina was 70% (P=0.01) (n=5). The diffusion of MPO, whose molecular weight is 70 kDa, into the vitreous humor was minimal in both groups of eyes. The plasma concentration of anti-ICAM-1 mAb, 64.5±73.4 ng/ml, was 31,000-fold less than the concentration in the osmotic pump.

The site through which VEGF was injected into the vitreous is unlikely to have served as a conduit for the mAb because experiments with FITC-IgG revealed no significant increase in intraocular concentration of fluorescence resulting from the creation of a scleral perforation at the pars plana, 20 mm distant from the pump tip. Furthermore, even if the perforation resulted in increased vitreous levels of mAb, it is unlikely to have had any impact upon the retinal or choroidal vasculature, owing to the diffusion barrier of the internal limiting membrane of the retina (Smelser, supra; Peyman, supra; Marmor, supra; Misono,

Statistics

Tissue concentrations of FITC-IgG were compared by standard linear analysis of variance, and the paired Student's t-test was used to compare MPO levels between eyes. All P values were two-tailed. An α level of 0.05 was used as the criterion to reject the null hypothesis of equality of means.

### Other Embodiments

All publications and patent applications mentioned in this specification are herein incorporated by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses or adaptations of the invention following, in general, the principles of the invention and including such departures from the present disclosure that come within known or customary practice within the art to which the invention pertains and may be applied to the essential features hereinbefore set forth.

1. A method of delivering a nucleic acid molecule into a mammalian eye, the method comprising contacting a scleral surface of the eye with a nucleic acid molecule having a molecular weight no greater than 150 kDa such that the nucleic acid passes through the sclera and into the interior of the eye.

2-20. (canceled)

21. The method of claim 1, wherein the nucleic acid has a molecular weight of at least 70 kDa.

22. The method of claim 21, wherein the nucleic acid has a molecular weight of at least 100 kDa.

23. The method of claim 22, wherein the nucleic acid has a molecular weight of at least 120 kDa.

24. A method of delivering a nucleic acid molecule into a mammalian eye, the method comprising contacting a scleral surface of the eye with a nucleic acid molecule having a molecular radius of at least 0.5 nm and a molecular weight no greater than 150 kDa so that the nucleic acid passes through the sclera and into the interior of the eye.

25. The method of claim 24, wherein the nucleic acid has a molecular radius of at least 3.2 nm.

26. The method of claim 24, wherein the nucleic acid has a molecular radius of at least 6.4 nm.

27. The method of claim 1 or 24, comprising the additional step of thinning the sclera prior to contacting the scleral surface with the nucleic acid.

28. The method of claim 27, wherein the sclera has a thickness less than 70% of its pre-thinned thickness.

29. The method of claim 28, wherein the sclera has a thickness less than 60% of its pre-thinned thickness.

30. The method of claim 1 or 24, wherein the nucleic acid is contacted with said sclera together with means for facilitating the transport of the nucleic acid through the sclera.

31. The method of claim 1 or 24, wherein the nucleic acid is delivered into contact with the scleral surface by a pump.

32. The method of claim 31, wherein the pump is a mechanical or osmotic pump.

33. The method of claim 1 or 24, wherein the nucleic acid is delivered into contact with the scleral surface by a microchip.

34. The method of claim 1 or 24, wherein the mammal is a human.

35. The method of claim 1 or 24, wherein the method is used to treat a retinal or choroidal disease.

36. The method of claim 35, wherein the retinal or choroidal disease is selected from the group consisting of macular degeneration, diabetic retinopathy, retinitis pigmentosa and other retinal degenerations, retinal vein occlusions, sickle cell retinopathy, glaucoma, choroidal neovascularization, retinal neovascularization, retinal edema, retinal ischemia, proliferative vitreoretinopathy, and retinopathy of prematurity.

37. The method of claim 1 or 24, wherein the nucleic acid molecule is a purified nucleic acid molecule.
